(19) United States
(12) Patent Application Publication
(10) Pub. No.: US 2018/0001085 A1
(43) Pub. Date: Jan. 4, 2018

(54) Handling and Control System for Expandable Electrodes of A Handpiece for Use in an Electro-Poration Process

(71) Applicant: IGEA S.p.A., Carpi (IT)

(72) Inventors: Ruggero Cadossi, Carpi (IT); Donata Marazzi, Carpi (IT); Claudio Bertacchini, Carpi (IT); Francesca De Terlizzi, Mogliano Veneto (IT); Mattia Ronchetti, Carpi (IT)

(73) Assignee: IGEA S.p.A., Capri (IT)

(21) Appl. No.: 15/638,849

(22) Filed: Jun. 30, 2017

(30) Foreign Application Priority Data
Jul. 1, 2016 (IT) 102016000068691

(51) Int. Cl. A61N 1/32; A61N 1/04

(52) U.S. Cl. CPC A61N 1/327; A61N 1/0412; A61N 1/0476

(57) ABSTRACT

A handling and control system for expandable electrodes of a handpiece is provided that includes a plurality of flexible electrodes made of elastic cables carried by a support assembly with needle-shaped front portions that protrude from the support assembly and move in a three-dimensional space under the push of actuators. An electronic control device performs the following functions: a) providing a command to the actuators to perform an initial handling of each cable according to an initial step Δh performing an axial advancement of the front portion with respect to the second proximal end and a distancing of the front portion from the axis H; b) determining for each pair of electrodes the spacing or distance lᵢ, measured along a direction perpendicular to the axis, between the tips of the front portions of the pair of electrodes; c) determining a voltage V as a function of the spacing lᵢ, V=f(lᵢ) and applying to each electrode a pulsed signal having maximum voltage equal to the calculated value V; e) repeating the steps a), b) and c) for a plurality n of steps k successive to the initial one so that the active portions of the electrodes move in space in a three-dimensional application area becoming distanced from each other; the voltage applied to the electrodes increasing linearly with the increasing of the spacing so as to generate an electric field which ensures in the application area complete electro-poration of tissue.

### PRIORITY CLAIM

This application claims priority from Italian Patent Application No. 102016000068691 filed on Jul. 1, 2016, the disclosure of which is incorporated by reference.

### TECHNICAL FIELD

The present invention relates to a handling and control system for expandable electrodes of a handpiece for use in an electro-poration process.

### BACKGROUND OF THE INVENTION

As it is known, the objective of the electro-poration technique of a tissue is to homogeneously expose all the cells contained in the tissue to an electric field having an intensity exceeding a local threshold value in order to obtain a permeabilizing effect on the cell membranes. In some applications (for example ablation of tumour tissues) this local threshold value is in the order of 400V/cm (in general for values greater than 250V).

The tissue to be subjected to electro-poration must be penetrated homogeneously by the electric field, which must have a value above the threshold value in the whole of the volume of application of the electric field.

In order to make this electric field spatially effective, in many therapeutic applications use pairs of needle-shaped electrodes, which are inserted into the tissue to be treated at the same depth, maintaining the parallelism between the needles. For example, electrodes with fixed geometry, i.e., provided with pairs of electrodes rigidly fixed to each other, can be used in order to ensure parallelism between the needles.

The requirements of parallelism between the electrodes and penetration uniformity are not easy to achieve when wishing to treat tumour nodules located in places that are difficult to reach, such as hollow or visceral organs. This requirement is difficult to apply when wishing to avoid open surgery, for example in surgical procedures for hepatobiliary and pancreatic tumours.

To treat the aforesaid localized tumours, ablation techniques (e.g. radiofrequency ablation) have been proposed that use a handpiece provided with an expandable beam of electrodes shaped in the form of an elastic cable provided with needle-shaped end portions.

The needle-shaped end portions are inserted into the tumour nodule and the electrodes are subsequently positioned and/or expanded in the tissue. The electromagnetic energy issued by the electrodes produces strong heating of the surrounding tissue causing degenerative coagulation of this tissue.

An example of a handpiece provided with expandable electrodes of the aforesaid type is described in the patent EP-B-2.032.057, which illustrates a support assembly comprising an insulating body elongated along an axis and defining at its inside a plurality of inner channels each of which extends from a first distal end of the elongated body for a straight portion parallel to the axis, which is contiguous and communicating with a second curved portion that has radial distance with respect to the axis, increasing towards a second portion of the proximal end of the elongated body. Each curved portion leads to the second proximal end through a respective opening. The handpiece further comprises a plurality of flexible electrodes (needles) carried by the support assembly and mobile with respect to said body under the action of a pushing system of manual type; each electrode comprises an elastic cable made of conductive material covered with an insulating sheath and provided with a front uncovered portion that forms a needle-shaped active portion. Each elastic cable is housed inside a respective channel with the active portion that, when in use, protrudes from the respective opening. The manual pushing system acts on the rear portion of the flexible cables to produce a movement of these cables along an advancement direction in which the front portions of the cables that protrude from the support assembly advance along the axis and simultaneously are radially distanced from this axis.

The electrodes of the aforesaid type do not guarantee parallelism between the needle-shaped active portions. Moreover, the expandable electrodes do not provide for adjustment of the value of the electric field applied as a function of the positioning, which is fixed; therefore, it is not possible to proceed with successive steps of electro-poration for segmentation of the treatment of the nodule.

For those reasons, expandable electrodes of known type do not ensure electro-poration of all the cells of the tumour nodule.

If the parallelism between the electrodes is not maintained, the electric field applied in V/cm may differ greatly between the nearest and farthest tips of the electrodes. For example, in the nearest tips they could have values (V/cm) such as to determine a discharge through the tissue, or in the farthest tips not reach the threshold value V.

### SUMMARY OF THE INVENTION

The object of the present invention is to produce a handling and control system for expandable electrode which also allows the use of a handpiece provided with expandable electrodes in an electro-poration process, ensuring complete electro-poration of a volume of tissue.

The aforesaid object is achieved by the present invention, as this relates to a system of the type described in claim 1.

### BRIEF DESCRIPTION OF THE DRAWINGS

The invention will now be illustrated with reference to the accompanying drawings that represent a preferred but non-limiting embodiment thereof, wherein:

FIG. 1 illustrates, in a schematic longitudinal section, a handling and control system for expandable electrodes of a handpiece produced according to the dictates of the present invention;

FIG. 2 illustrates handling and control operations of the expandable electrodes produced according to the present invention; and

FIG. 3 schematizes the use of the expandable electrode;

FIG. 4 illustrates a first variant of the system of FIG. 1; and

FIG. 5 illustrates a second variant of the system of FIG. 1.

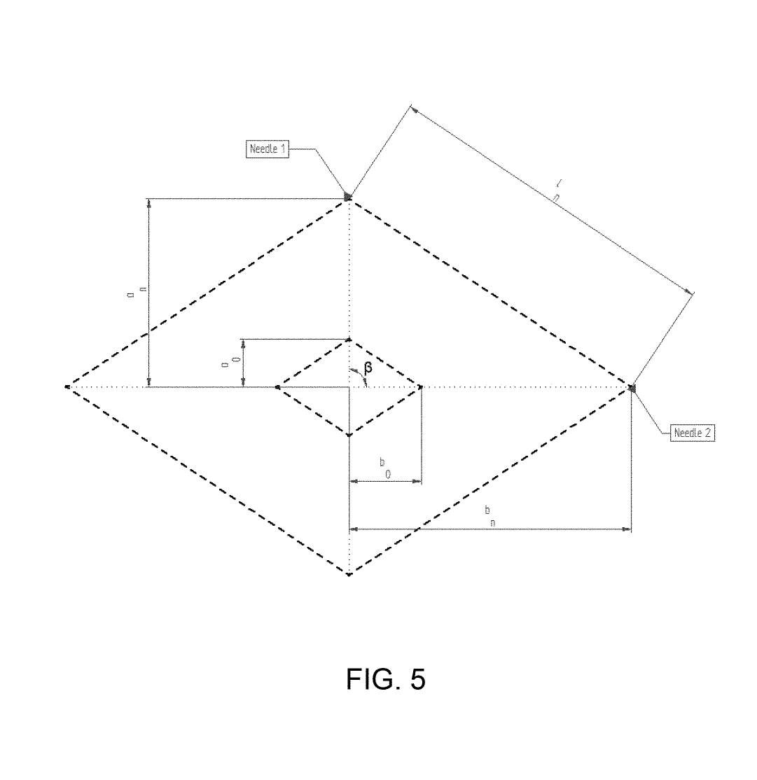

### DETAILED DESCRIPTION OF THE INVENTION

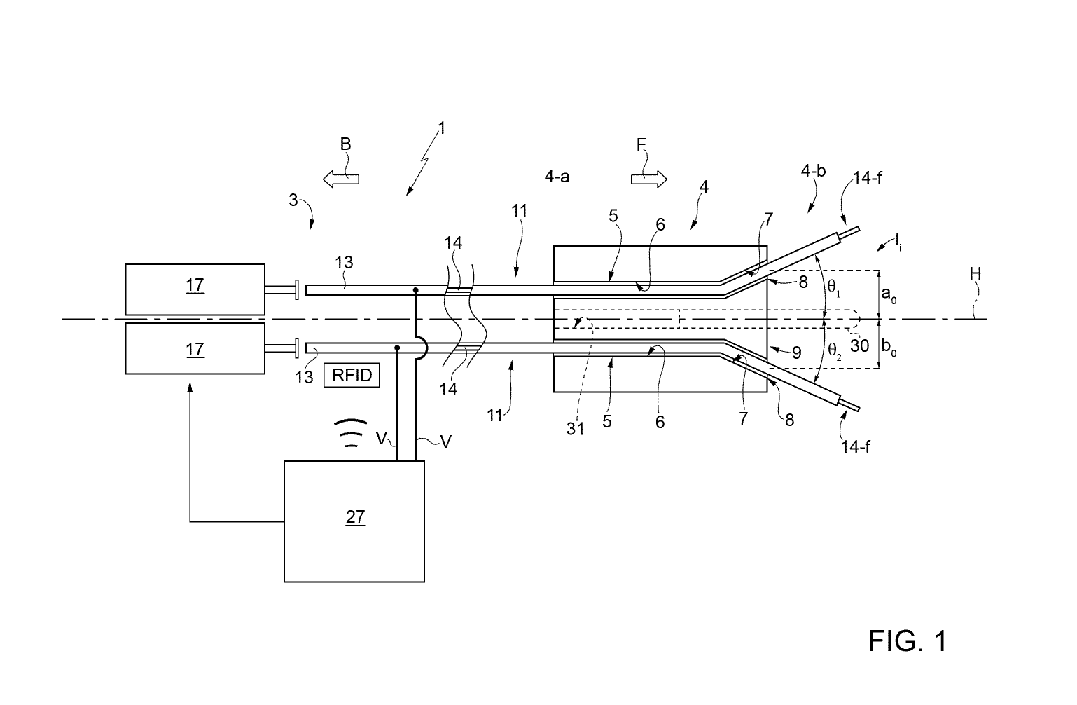

FIG. 1 indicates with 1, in its entirety, a handling and control system for handpiece 2 provided with expandable electrodes (illustrated schematically). The handpiece 2 can advantageously be used in an electro-poration process that does not form part of the subject-matter of this patent application, which only addresses the electrical, mechanical and control aspects of the system and the structure of the handpiece and of the electrodes.

The handpiece 2 comprises a support assembly—graspable electrodes 3 (which can be rigid or flexible—this graspable element is illustrated partially and schematically) provided with an elongated insulating body 4 along an axis H at one of its end (in the example a cylindrical body but the shape may vary) and defining at its inside a plurality of inner channels 5 (in the example channels with circular cross section) each of which extends from a first distal end 4-a of the elongated body 5 for a first straight portion 6 parallel to the axis H which is contiguous and communicating with a second straight portion 7 that extends along a direction which forms a divergence angle θ with respect to the axis H (and therefore also to the direction of extension of the first straight portion) and therefore has a radial distance with respect to the axis H increasing towards a second portion of the proximal end 4-b of the elongated body 4.

Typically, the divergence angle θ is variable from 5 to 45 degrees.

Each second straight portion 7 leads to the second proximal end 4-b through a respective opening 8 that opens in a wall 9 (in the example a flat wall perpendicular to the axis H but the shape could may vary) of this second portion of proximal end portion 4-b.

The handpiece 2 comprises a plurality of flexible electrodes 11 (to simplify the description, only one pair of electrodes is illustrated, but the number of electrodes can differ and form, for example, two, three, four or more pairs) carried by the support assembly 3 and by the elongated insulating body 4 and mobile with respect to the elongated insulating body 4 under the action of a pushing system 12 (illustrated schematically).

Each electrode 11 of the pair comprises a flexible cable 14 made of elastic conductive material (for example a steel wire) covered with an insulating sheath 13 and provided with a needle-shaped uncovered front portion 14-f (length comprised between 2 mm and 4 cm) that forms an active portion.

Each electrode 11 is housed inside a respective channel 5 with the active portion 14-f that, when in use, protrudes from the respective opening 8 positioned at the end of the channel 5.

The pushing system 12 acts, for example, on the rear portion 14-b of the elastic cables 14 to perform a movement along an advancement direction F of these cables between a position at rest (not illustrated) in which the flexible electrodes are housed inside the respective channels 5 (and therefore the needle-shaped portions are not accessible ensuring safety) and a using position (FIG. 1) in which the front portions 14-f of the cables 14 and the electrode portions adjacent thereto, protrude from the support assembly 3.

The movement of the cables 14 along the advancement direction F produces the movement of the front portions 14-f along the axis H, axial distancing of these front portions 14-f from the second end 4-b and the radial distancing of the portions 14-f from the axis H due to the presence of the second rectilinear portions 7 that cause the advancement of the elastic cables along a direction inclined by the angle θ with respect to the axis H.

The portion of the elastic cables 14 that protrude from the openings 8 extends substantially rectilinearly due to the guide effect provided by the second straight portion 7. The protruding rectilinear portion thus forms an angle θ with respect to the axis H.

The movement of the cables 14 along a retraction direction B opposite the advancement direction F produces a movement of the front portions 14-f towards the second proximal end portion 4-b and radial movement of the portions 14-f towards the axis H. This movement continues until the portions 14-f return inside their respective channels 5 and the electrodes 11 are contained inside the support assembly 3. This position allows the electrodes to be kept safely, preventing accidental contact between the needle-shaped portions and an operator (not illustrated) handling the handpiece 2.

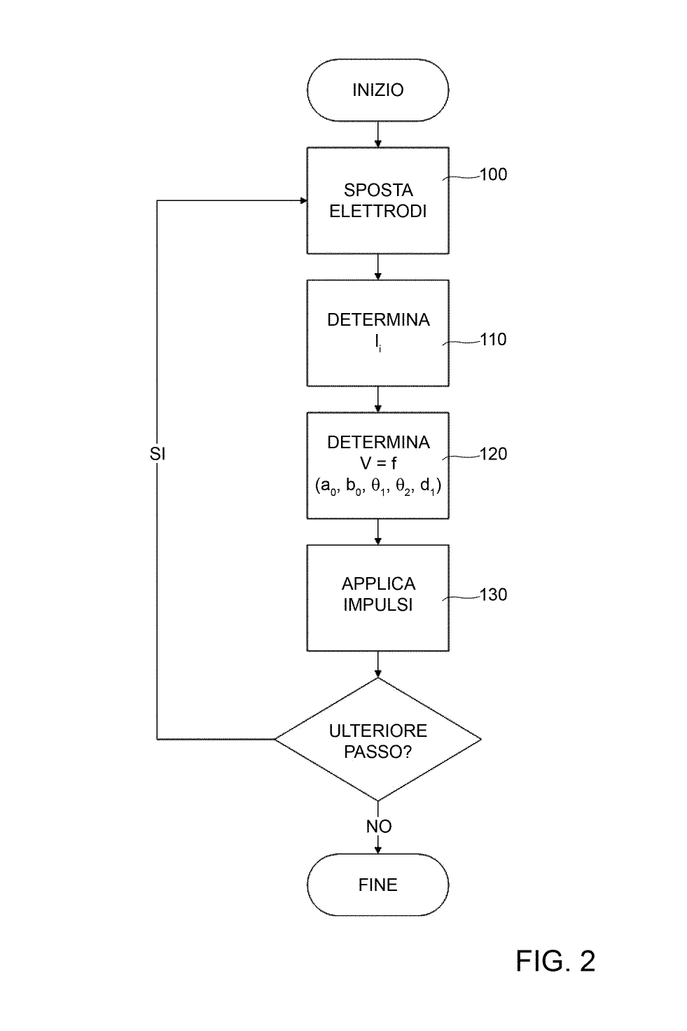

According to the present invention, there is provided an electronic control device 27 that controls the actuators 17 of the pushing system 12 and the voltage V applied to the electrodes 11 performing the operations described with reference to the flow-chart of FIG. 2.

The following operations are performed:

a) providing a command to the actuators 17 (in the example electromechanical actuators but one actuator could also be of a mechanical type) to perform an initial handling (block 100) of each cable 14 of the pair of electrodes 11 according to an initial pitch Δh performing an axial advancement of the front portion 14-f with respect to the second proximal end 4-b and a distancing of the front portion 14-f from the axis H,
b) determining for each electrode 11 of the pair the spacing or distance measured along a direction perpendicular to the axis H, between the tips of the front portions 14-f (block 110) of the pair of electrodes 11 (spacing between tips);
c) determining a voltage V (block 120) as a function of the spacing and applying to the electrodes 11 of the pair a pulsed signal (block 130) having maximum voltage V—the pulsed signal can have a different waveform (square, triangular, sinusoidal, sawtooth, etc.) and can have a fixed or variable frequency;
d)
repeating the above steps a), b) e c) for a plurality k of steps k1, k2, ki, . . . kn successive to the initial one and therefore for a plurality of increasing steps Δh1 Δh2 . . . Δhi . . . Δhn and increasing spacing values l1, l2 . . . lᵢ . . . ln between the electrodes of the pair so that the active portions 14-f of the electrodes 11 of the pair move in space in a three-dimensional application area; the voltage applied to the needle portions 14-f of the pair of electrodes 11 varies as a function of the spacing between the same electrodes increasing linearly with the increasing of the spacing.

Preferably the relation V=f(lᵢ) between voltage V and lᵢ is mapped in a table that outputs different increasing output values V for increasing values according to a linear law, for example those indicated in the table below:

| | |
|---|---|
| l1 = 1 cm | V1 = 1000V |
| l2 = 2 cm | V2 = 2000V |
| L3 = 3 cm | V3 = 3000V |
| Li | Vi |
| Ln | Vn |

The n number k of steps can be set manually in order to define the maximum displacement of the active portions 14-f of the electrodes 11 with respect to the position taken by the active portions 14-f prior to the initial handling and consequently define a spacing I_{MAX} between the tips of the active portions 14-f of the electrodes 11 of the pair. For example, the maximum spacing can take the value of 3 cm; a maximum voltage of 3000V is applied at this maximum spacing).

The spacing lᵢ is determined based on the radial distance a₀, b₀ between the outlet opening 8 of each second straight portion 7 of the pair of electrodes 11 and the axis H, according to the divergence angles θ1 e θ2 that each second straight portion 7 forms with respect to the axis H and according to the distance measured/estimated along the axis H between the wall 9 and the plane perpendicular to the axis H on which rest the tips of the end portions 14-f.

Typically, the following relation can be used:

lᵢ=a₀, +b₀₊dᵢ tan(θ1)+dᵢ tan(θ2).

where the distance dᵢ corresponds to the insertion depth of the electrodes measured along the axis H when the wall 9 is arranged in contact with a portion of human body and the electrodes 11 are inserted in this portion of human body.

The distance dᵢ can be determined indirectly by means of a mapping that reports for each step a respective value dᵢ, namely

| | |
|---|---|
| k1 | d1 = 5 mm |
| | with (a₀ = b₀ = 2 mm, θ1 = θ2 = 45°) |
| k2 | d2 = d1 + 5 mm |
| . . . | |
| Ki | di = d(i − 1) + 5 mm |
| . . . | |
| Kn | dn = d(n − 1) + 5 mm |

Or can be determined directly by means of a sensor that measures the axial displacement of a rectilinear central electrode 30 (indicated with a dashed line) mobile along the axis H and housed in an axial cavity 31 of the elongated insulating body.

These relations are used by the block 110 to determine dᵢ.

In any case, the electronic control device 27 is configured to perform steps Δh having a length smaller than the length of the front uncovered needle-shaped portion 14-f.

The maximum length of the active part can be selected as a function of the divergence angle θ°, so that the length decreases as the angle increases and the electric field maintains a significant homogeneity in the volume affected by the electro-poration.

For example, according to the values contained in the attached table:

| Divergence angle: θ° | Active part mm |
|---|---|
| 5 | 18.0 |
| 10 | 9.1 |
| 15 | 6.2 |
| 22.5 | 4.3 |
| 45 | 3.0 |

the information relating to the values of a₀, b₀, at the divergence angles θ1 and θ2 and at the values taken by dᵢ for successive steps can be memorized—for each electrode or for each pair of electrodes—in the memory associated with an RFID device (FIG. 1) which is automatically activated when arranged in proximity of the electronic unit 27 to download the information a₀, b₀, θ1 and θ2 and dᵢ (if present in mapped form) and allow calculation of V. The RFID device could also provide other information, for example number of the pairs of electrodes 11 and length of the active portion 14-f.

Repetition of the steps a), b) e c) ensures that, for each position of the electrodes 11 in the three-dimensional application area, the potential V applied to each pair of electrodes, is updated so that the local value of the electric field is higher than the set threshold value thus obtaining the desired permeabilization effect on the cell membranes of the area of tissue corresponding to the three-dimensional application area.

When in use, the proximal end portion 4-b rests on or is placed in proximity to a portion of human body with the electrodes 11 arranged in the rest position. The electrodes 11 are then extracted and made to penetrate the tissues of the human body (naturally with the patient under local or total anaesthetic) until the needle-shaped portions 14-f reach a portion of tissue to be treated (for example a tumour nodule). During these positioning operations, no voltage is applied to the electrodes 11. The central electrode 30—if present—also moves along the axis H and contributes to the measurement of the insertion depth dᵢ.

Subsequently, the electronic unit 27 takes control of the expandable electrode 2 and a voltage V is calculated/applied according to the values a₀, b₀, θ1 and θ2 (discharged by means of the use of RFID) and according to the value dᵢ measured or estimated

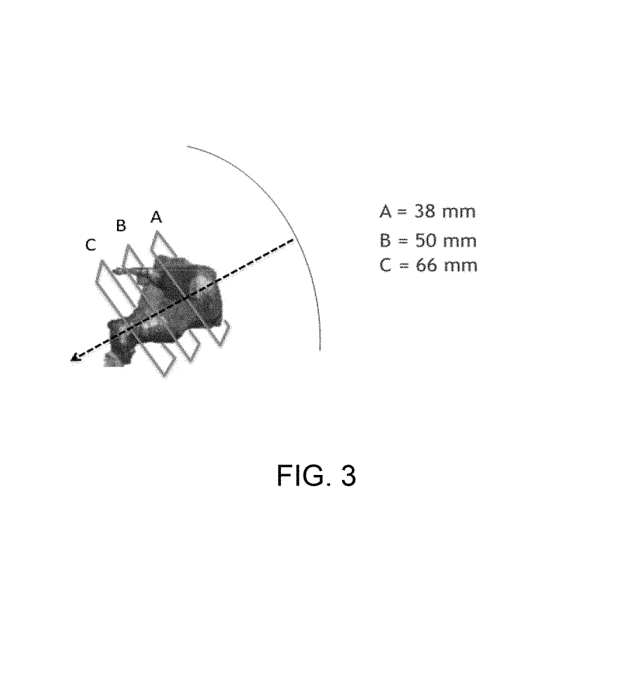

A signal having maximum voltage V is applied to each pair of electrodes 11 for a preset time interval, and electro-poration of a “slice” A of tumour nodule (see FIG. 3) is performed with an electric field that has a value locally above the threshold. As stated above, the voltage value applied is calculated so that it has a suitable value according to the spacing between the electrodes 11 and the thickness of which depends on the length of the active part of the needle 14-f (FIG. 3).

The actuator 17 applies to the electrodes 11 a first displacement Δh performing an axial advancement Δr of the front portion 14-f inside the nodule, the process to calculate the block 120 is repeated, and electro-poration of a second slice of nodule (slice “B”, once again see FIG. 3) is performed with linearly increasing voltage. The spacing between the active portions 14-f of the electrodes is in fact increased and it is necessary to apply a higher voltage to ensure that the portion of the tissue arranged between the electrodes is penetrated by an electric field having a value above the threshold in each area of the nodule.

The actuator 17 applies to the electrodes 11 a second displacement Δh performing a further axial advancement Δr of the front portion 14-f inside and around the nodule, the calculation process of the block 120 is repeated, and electro-poration of a third slice of nodule (slice “C”) is performed.

Repetition of the aforesaid operations allows electro-poration of N slices (for example ten slices, of the tumour nodule.

Following each “push” (i.e. displacement Δh) the electric field is “adjusted”, namely recalculated as a function of the new positioning of the needle-shaped portions 14-f inside and around the tumour nodule. The process is repeated until the whole of the nodule (its depth) has been subjected to electro-poration.

If three electrodes 11 are provided housed inside the respective channels 5 that lead to the wall 9 through respective openings whose centres are arranged at the vertices of a triangle and form, one with respect to those adjacent thereto and with respect to the trace of the axis H, an angle β (beta) measured on a plane perpendicular to the axis H,

the electronic unit 27 determines the spacing lᵢ between each pair of electrodes belonging to the group of three electrodes on the basis of:

lᵢ=\sqrt{aₙ²+bₙ²−2aₙbₙ cos β}

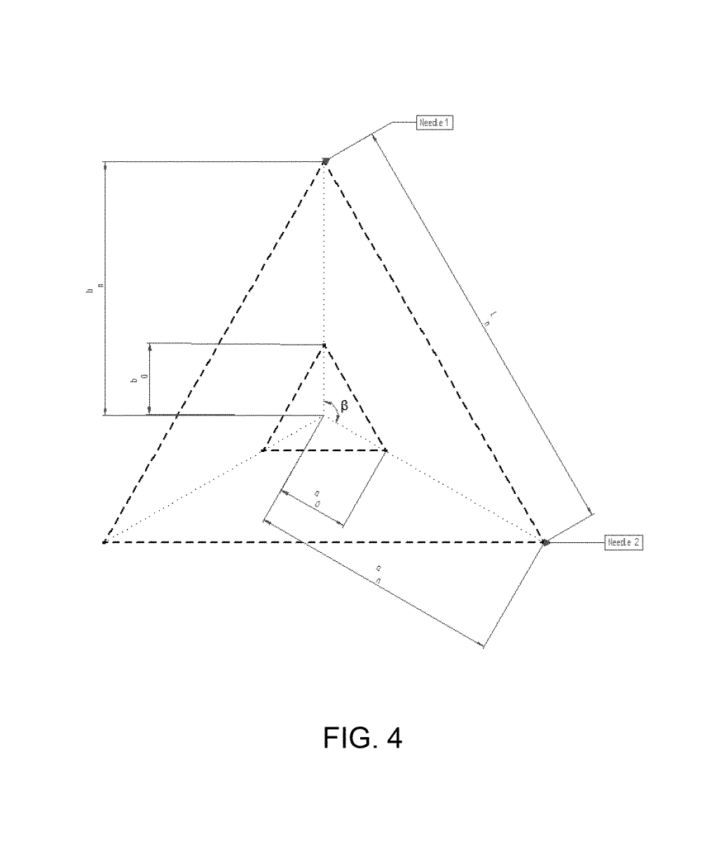

with
aₙ=a₀, +dₙ tan(θ1)
bₙ=b₀, +dₙ tan(θ2)
where a represents the radial distance between the centre of the opening 8 of a first electrode of the pair and the trace of the axis H, and b represents the radial distance between the centre of the opening 8 of a second electrode of the pair and the trace of the axis H. (FIG. 4).

If four electrodes 11 are provided housed inside respective channels 5 that lead onto the wall 9 through respective openings whose centres are arranged at the vertices of a rhombus and form, one with respect to those adjacent thereto and with respect to the trace of the axis H, an angle β (beta) measured on a plane perpendicular to the axis H,

the electronic unit 27 determines the spacing lᵢ between each pair of electrodes belonging to the group of four electrodes on the basis of:

lᵢ=\sqrt{aₙ²+bₙ²−2aₙbₙ cos β}

with
aₙ=a₀, +dₙ tan(θ1)
bₙ=b₀, +dₙ tan(θ2)
where a represents the radial distance between the centre of the opening 8 of a first electrode of the pair and the trace of the axis H, and b represents the radial distance between the centre of the opening 8 of an adjacent second electrode of the pair and the trace of the axis H.

1. A handling and control system for expandable electrodes (2) of a handpiece for use in an electro-poration process comprising:
a support assembly (3) provided with an elongated insulating body (4) along an axis H at one of its ends and defining in its inside a plurality of inner channels (5) each of which extends from a first distal end (4-a) of the elongated body (4) for a straight portion (6) parallel to the axis H which is contiguous and communicating with a second straight portion (7) that extends along a direction which forms a divergence angle with respect to the axis H having a radial distance with respect to the axis H increasing towards a second portion of the proximal end (4-b) of the elongated body;
each second straight portion (7) leads to the second proximal end (4-b) through a respective opening (8);
a plurality of flexible electrodes (11) carried by said support assembly and mobile with respect to said body under the action of a pushing system (12); each electrode comprising an elastic cable (14) made of conductive material covered with an insulating sheath (13) and provided with a front uncovered portion (14-f) that forms a needle-shaped active portion;
each elastic cable (14) being housed inside a respective channel (5) with the active portion (14-f) that, when in use, protrudes from the respective opening (8);
the pushing system (12) acting on the flexible cables (14) to perform a movement of the cables themselves along an advancement direction (F) wherein the front portions of the cables (14) that protrude from the support assembly advance along said axis H and at the same time radially distance themselves from the axis H itself;
the handling and control system (2) being characterized in that it further comprises an electronic control device (27) that controls the actuators (17) of the pushing system and the voltage applied to the electrodes performing the following functions:
a) providing a command to the actuators (17) to perform an initial handling of each cable (14) according to an initial step Δh, performing an axial advancement of the front portion (14-f) with respect to the second proximal end (4-b) and a distancing of the front portion (14-f) from the axis H;
b) determining for each pair of electrodes (11) the spacing measured along a direction perpendicular to the axis (H), between the tips of the front portions (14-f) of the pair of electrodes (11);
c) determining a voltage V as a function of said spacing lᵢ, V=f(lᵢ) and applying to each electrode a pulsed signal having maximum voltage equal to the determined voltage V;
d) repeating the above steps a), b) and c) for a plurality n of steps k successive to the initial one so that the active portions (14-f) of the electrodes move in space in a three-dimensional application area becoming distanced from each other; the voltage applied to the electrodes increases with the increasing of the spacing according to a set law and is such as to generate an electric field which ensures in said application area the complete electro-poration of the tissue.

2. The system according to claim 1, wherein said electronic control device (27) is configured to determine said spacing lᵢ based on the radial distance a₀, b₀ between the outlet opening (8) of each second straight portion (7) of the pair of electrodes (11) and the axis (H), according to the divergence angles θ1 and θ2 that each second straight portion (7) form with respect to the axis H and according to the distance measured or estimated along the axis H between an end wall (9) of the second proximal end (4-b) and the plane perpendicular to the axis H on which rest the tips of the end portions (14-f).

3. The system according to claim 2, wherein said electronic control device (27) is configured with at least two electrodes for determining said spacing lᵢ according to the following relationship:
lᵢ=a₀, +b₀₊dᵢ tan(θ1)+dᵢ tan(θ2).

4. The system according to claim 2, wherein a RFID device is provided which is automatically activated when arranged in proximity to the electronic unit (27) to download to the electronic unit (27) the information associated with at least a₀, b₀ and θ1 and θ2.

5. The system according to claim 2, wherein sensors for directly detecting the value of dᵢ are provided.

6. The system according to claim 5, wherein said sensors comprise a sensor that measures the axial displacement dᵢ of a rectilinear central electrode (30) mobile along the axis H and housed in a central axial cavity (31) of the elongated insulating body (4).

7. The system according to claim 2, wherein said electronic control device (27) is configured to determine dᵢ indirectly by means of a mapping that reports, for each step k, a respective value of dᵢ.

8. The system according to claim 1, wherein said electronic unit (27) is configured to set a number k of steps in order to define the maximum spacing between the active portions (14-f) of the electrodes (11).

9. The system according to claim 1, wherein said electronic control device (27) is configured to perform steps, Δh having a length smaller than the length of the front uncovered needle-shaped portion (14-f).

10. The system according to claim 1, wherein three electrodes (11) are provided, housed inside respective channels (5) leading to an end wall (9) of the second proximal end portion (4-b) through respective openings whose centres form, one with respect to those adjacent thereto and with respect to the trace of the axis H, an angle β (beta) measured on a plane perpendicular to the axis H;
the electronic unit (27) is configured to determine the spacing between each pair of electrodes belonging to the group of three electrodes on the basis of:
with
lᵢ=\sqrt{aₙ²+bₙ²−2aₙbₙ cos β}
with
aₙ=a₀, +dₙ, tan(θ1)
bₙ=b₀, +dₙ tan(θ2)
where a represents the radial distance between the centre of the opening (8) of a first electrode of the pair and the trace of the axis H, and b represents the radial distance between the centre of the opening (8) of a second electrode of the pair and the trace of the axis H.

11. The system according to claim 1, wherein at least four electrodes are provided housed inside respective channels (5) which lead to an end wall (9) of the second portion of the proximal end (4-b) through respective openings whose centres are arranged at the vertices of a polygon and form, one with respect to those adjacent thereto and with respect to the trace of the axis H, an angle β (beta) measured on a plane perpendicular to the axis H,
the electronic unit (27) determines the spacing between each pair of electrodes belonging to the group of four or more electrodes on the basis of:
lᵢ=\sqrt{aₙ²+bₙ²−2aₙbₙ cos β}
with
aₙ=a₀, +dₙ tan(θ1)
bₙ=b₀, +dₙ tan(θ2)
where a represents the radial distance between the centre of the opening 8 of a first electrode of the pair and the trace of the axis H, and b represents the radial distance between the centre of the opening 8 of an adjacent second electrode of the pair and the trace of the axis H.
